(19) United States
(12) Patent Application Publication
(10) Pub. No.: US 2011/0224487 A1
(43) Pub. Date: Sep. 15, 2011

(54) IMAGE PICKUP MODULE, MANUFACTURING METHOD THEREOF, AND ENDOSCOPIC DEVICE

(75) Inventor: Teppei OGAWA, Saitama-shi (JP)

(73) Assignee: FUJIFILM CORPORATION, Tokyo (JP)

(21) Appl. No.: 13/013,167

(22) Filed: Jan. 25, 2011

(30) Foreign Application Priority Data
Mar. 15, 2010 (JP) 2010-057869

(51) Int. Cl. A61B 1/04; H01L 31/0203; H01L 31/18

(52) U.S. Cl. 600/109; 257/434; 438/64; 257/E31.118; 257/E31.12

(57) ABSTRACT

Size reduction of an image pickup module is promoted, and reliability of electric connection and electric noise resistance are improved by decreasing the numbers of components and connection spots. The problems are solved by providing an image pickup module including a solid-state image pickup element chip having an image pickup surface, a cover glass that covers the image pickup surface, and a wiring board on which the solid-state image pickup element chip is mounted, in which the solid-state image pickup element chip and the wiring board have an overlap structure in which end portions thereof are overlapped with each other, and a first electrode portion formed on the end portion of the solid-state image pickup element chip and a second electrode portion formed on the end portion of the wiring board are electrically connected through a bump.

### BACKGROUND OF THE INVENTION

1. Field of the Invention

The presently disclosed subject matter relates to an image pickup module, a manufacturing method thereof, and an endoscopic device and particularly to a technology to mount a solid-state image pickup element chip on a flexible board.

2. Description of the Related Art

In the medical field, diagnoses using an endoscopic device (electronic endoscope) are widely performed. The endoscopic device includes an insertion portion to be inserted into a body cavity of a patient (subject body) and an operation portion consecutively connected to a base end of the insertion portion. In a distal end portion of the insertion portion, an image pickup module (image pickup device) having a solid-state image pickup element such as a CCD image pickup element and a CMOS image pickup element is incorporated.

In such an endoscopic device, in order to make insertion into the patient smooth and to alleviate a burden on the patient as well as an operator who operates the endoscope, size reduction of the image pickup module is in demand.

For example, Japanese Patent Application Laid-Open No. 2008-34505 discloses a solid-state image pickup device in which an end portion of a flexible board is bonded to a side face of a solid-state image pickup element chip so that an electrode pad disposed on the surface of the solid-state image pickup element chip and an electrode for connection composed of a thick part of an internal wiring pattern formed on an end face of the flexible board are disposed substantially on the same plane, and the electrode pad of the solid-state image pickup element chip and the electrode for connection of the flexible board are electrically connected to each other by wire bonding using a wire.

Also, Japanese Patent Application Laid-Open No. 8-172177 discloses a solid-state image pickup module provided with a glass board in which a wiring circuit including a required input/output connection terminal is disposed on one principal surface, solid-state image pickup elements whose light receiving faces are mounted and arranged to face each other on the one principal surface of the glass board, a connection portion that electrically connects a terminal of the solid-state image pickup element to one of the connection terminals of the glass board face, an active circuit element interposed in the wiring circuit of the glass board face, and a flexible wiring board that electrically connects to the other connection terminal of the glass board face.

### SUMMARY OF THE INVENTION

However, with the structure disclosed in Japanese Patent Application Laid-Open No. 2008-34505, since the electrode pad of the solid-state image pickup element chip and the electrode for connection of the flexible board are electrically connected to each other by wire bonding using a wire, the number of components is large, and deterioration of connection reliability caused by an increase of connection spots is concerned about.

Also, with the structure disclosed in Japanese Patent Application Laid-Open No. 8-172177, since the glass board is interposed in the connection between the solid-state image pickup element and the flexible wiring board, it is disadvantageous for the size reduction of the image pickup module and the electric noise for the portion of the wiring on the glass board. Also, similarly to the structure disclosed in Japanese Patent Application Laid-Open No. 2008-34505, there is a concern about the deterioration of connection reliability caused by an increase of connection spots.

The presently disclosed subject matter was made in view of the above circumstances and has an object to provide an image pickup module, a manufacturing method thereof, and an endoscopic device in which the size reduction of the image pickup module is promoted, and the reliability of electric connection and electric noise resistance are improved by decreasing the numbers of components and the connection spots.

In order to achieve the above object, an image pickup module according to the presently disclosed subject matter, includes: a solid-state image pickup element chip having an image pickup surface; a cover glass that covers the image pickup surface; a wiring board on which the solid-state image pickup element chip is mounted; a overlap portion in which an end portion of the solid-state image pickup element chip and an end portion of the wiring board overlap each other; and a connection terminal portion configured to electrically connect a first electrode portion formed on the end portion of the solid-state image pickup element chip and a second electrode portion formed on the end portion of the wiring board through a bump.

According to the presently disclosed subject matter, the module has an overlap structure (overlap portion) in which the end portions of the solid-state image pickup element chip and the wiring board overlap each other, and the first electrode portion formed on the end portion of the solid-state image pickup element chip and the second electrode portion formed on the end portion of the wiring board are electrically connected to each other through the bump. That is, the solid-state image pickup element chip has an extended arrangement structure extending outward from the end portion of the wiring board, and the solid-state image pickup element chip and the wiring board are electrically connected directly without interposing an intermediate connecting member between them (such as a wire by wire bonding, a board for connection and the like). As a result, the size reduction of the image pickup module can be promoted, and the reliability of the electric connection and electric noise resistance are improved by reduction of the numbers of components and the connection spots.

In the presently disclosed subject matter, the end portions of the solid-state image pickup element chip and the wiring board are preferably sealed and fixed by a resin. Connection strength between the solid-state image pickup element chip and the wiring board can be ensured, and reliability of the electric connection can be improved.

Also, in the presently disclosed subject matter, the first electrode is preferably formed on a same plane as the image pickup surface. As compared with the case in which the first electrode of the solid-state image pickup element chip is formed on a plane different from the image pickup surface, the size reduction of the image pickup module can be improved. Also, a wiring length from the image pickup surface to an electrode pad can be reduced, and the electric noise resistance can be also improved.

Also, in the presently disclosed subject matter, the wiring board is preferably a flexible board having flexibility, and the flexible board more preferably includes at least a base layer, a wiring pattern formed on the base layer, and a cover layer that covers the surface of the base layer on which the wiring pattern is formed.

Also, in order to achieve the above object, a manufacturing method of the image pickup module according to the presently disclosed subject matter, the manufacturing method of an image pickup module including a solid-state image pickup element chip having an image pickup surface, a cover glass that covers the image pickup surface, and a wiring board on which the solid-state image pickup element chip is mounted and includes: aligning an end portion of the solid-state image pickup element chip and an end portion of the wiring board to overlap each other; and electrically connecting, after the alignment, a first electrode portion formed on the end potion of the solid-state image pickup element chip and a second electrode portion formed on the end portion of the wiring board through a bump.

According to the presently disclosed subject matter, the module has an overlap structure (overlap portion) in which the end portions of the solid-state image pickup element chip and the wiring board overlap each other, and the first electrode portion formed on the end portion of the solid-state image pickup element chip and the second electrode portion formed on the end portion of the wiring board are electrically connected to each other through the bump. That is, the solid-state image pickup element chip has an extended arrangement structure extending outward from the end portion of the wiring board, and the solid-state image pickup element chip and the wiring board are electrically connected directly without interposing an intermediate connecting member between them (such as a wire by wire bonding, a board for connection and the like). As a result, the size reduction of the image pickup module can be promoted, and the reliability of the electric connection and electric noise resistance are improved by reduction of the numbers of components and the connection spots.

Also, the numbers of components and connection spots for electric connection between the wiring board and the solid-state image pickup element chip are small, and mounting/assembling workability of the image pickup module is improved.

In the presently disclosed subject matter, the manufacturing method preferably further includes: applying a thermosetting resin on at least one of the end portions of the solid-state image pickup element chip and the wiring board; and heating and curing the thermosetting resin in a state in which the first electrode portion and the second electrode portion are electrically connected to each other through the bump. The connection strength between the solid-state image pickup element chip and the wiring board can be easily ensured, and reliability of the electric connection can be improved.

In the presently disclosed subject matter, the heating of the thermosetting resin is preferably performed indirectly through a tool that adsorbs the solid-state image pickup element chip. Also, the thermosetting resin is preferably a resin that is cured at 180 degrees or below, because thermal deterioration of the solid-state image pickup element chip can be effectively prevented.

Also, in order to achieve the above object, the endoscopic device according to the presently disclosed subject matter is characterized by including an image pickup module to which the presently disclosed subject matter is applied. As a result, the size of the insertion portion into which the image pickup module is incorporated can be reduced, and a burden on a patient or an operator can be alleviated.

According to the presently disclosed subject matter, the module has an overlap structure (overlap portion) in which the end portions of the solid-state image pickup element chip and the wiring board overlap each other, and the first electrode portion formed on the end portion of the solid-state image pickup element chip and the second electrode portion formed on the end portion of the wiring board are electrically connected to each other through the bump. That is, the solid-state image pickup element chip has an extended arrangement structure extending outward from the end portion of the wiring board, and the solid-state image pickup element chip and the wiring board are electrically connected directly without interposing an intermediate connecting member between them (such as a wire by wire bonding, a board for connection and the like). As a result, the size reduction of the image pickup module can be promoted, and the reliability of the electric connection and electric noise resistance are improved by reduction of the numbers of components and the connection spots.

### BRIEF DESCRIPTION OF THE DRAWINGS

FIG. 1 is an entire configuration diagram illustrating an electronic endoscopic system;

FIG. 2 is a perspective view illustrating a configuration of a distal end portion of an insertion portion;

FIG. 3 is an outline diagram illustrating an essential part of an internal structure of the distal end portion;

FIGS. 4A and 4B are configuration diagrams illustrating a detail of an image pickup module incorporated in the distal end portion; and

FIGS. 5A to 5D are explanatory diagrams illustrating an example of a manufacturing method of the image pickup module.

### DETAILED DESCRIPTION OF THE PREFERRED EMBODIMENTS

A preferred embodiment of an image pickup module and a manufacturing method thereof according to the presently disclosed subject matter will be described below in detail according to the attached drawings.

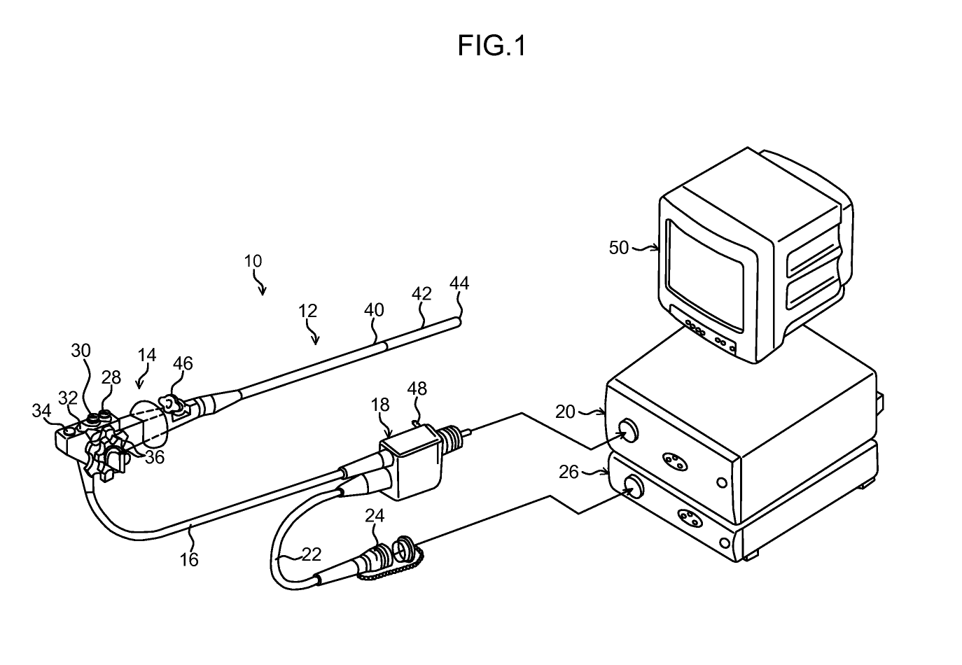

FIG. 1 is an entire configuration diagram illustrating an endoscopic system. The endoscopic system shown in FIG. 1 mainly includes an endoscopic device (electronic endoscope) 10 on which an image pickup module to which the presently disclosed subject matter is applied is mounted, a processor 26, a light source device 20, and a monitor device 50.

The endoscopic device 10 mainly includes an insertion portion 12 to be inserted into a body cavity of a patient (subject) and a hand operation portion 14 consecutively connected to a base end portion of the insertion portion 12.

In the hand operation portion 14, an air/water feed button 28, a suction button 30, a shutter button 32, a function switching button 34, and a pair of angle knobs 36 and 36 are disposed. Also, a forceps inlet 46 through which a treatment instrument such as forceps are inserted is disposed.

Also, in the hand operation portion 14, an LG connector 18 is disposed through a universal cable 16, and the LG connector 18 is detachably joined to a light source device 20. Also, to the LG connector 18, an electric connector 24 is connected through a cable 22, and the electric connector 24 is detachably joined to a processor 26.

The insertion portion 12 is composed of a distal end portion 44, a bent portion 42, and a flexible portion 40 in the order from the distal end side (opposite the hand operation portion 14).

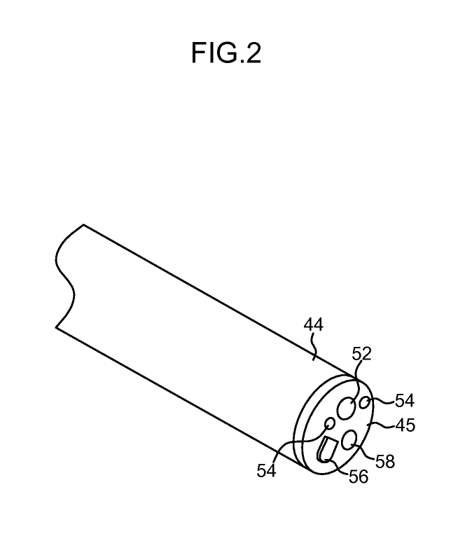

In the distal end portion 44 consecutively connected to the distal end of the insertion portion 12, as shown in FIG. 2, an observation window 52 that takes in subject light (reflection light from a portion to be observed) is disposed. Also, illumination widows 54 and 54 that radiate illumination light sent through the universal cable 16 or the like from the light source device 20 to the subject, an air/water feed nozzle 56 that injects washing water or air for washing off stains on the observation window 52 by operating the air/water feed button 28, and a forceps outlet 58 communicating with the forceps inlet 46 are disposed.

On the base end side (hand operation portion 14 side) of the distal end portion 44, the bent portion 42 in which a plurality of bent pieces are connected is disposed. The bent portion 42 is bent and operated vertically and horizontally by pushing/pulling a wire inserted and installed in the insertion portion 12 in conjunction with the operation of the angle knobs 36 and 36 disposed on the hand operation portion 14. As a result, the distal end portion 44 is directed to a desired direction in the subject.

On the base end side of the bent portion 42, a flexible portion 40 having flexibility is disposed. The flexible portion 40 has a length of one to several meters so that the distal end portion 44 can reach the portion to be observed and that a distance from a patient is kept to such a degree that an operator can grasp and operate the hand operation portion 14 without trouble.

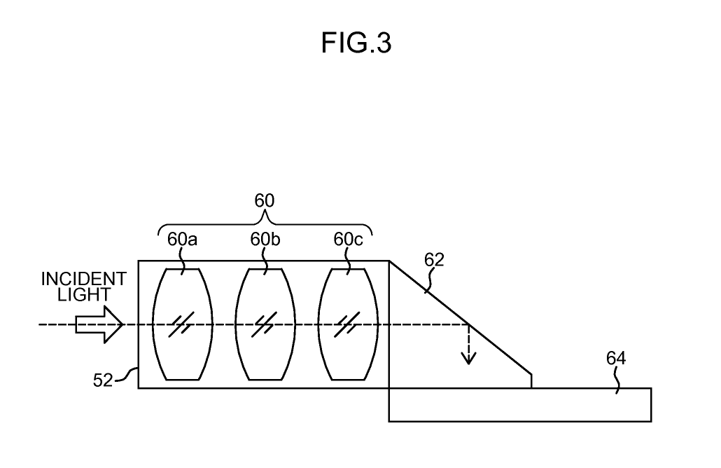

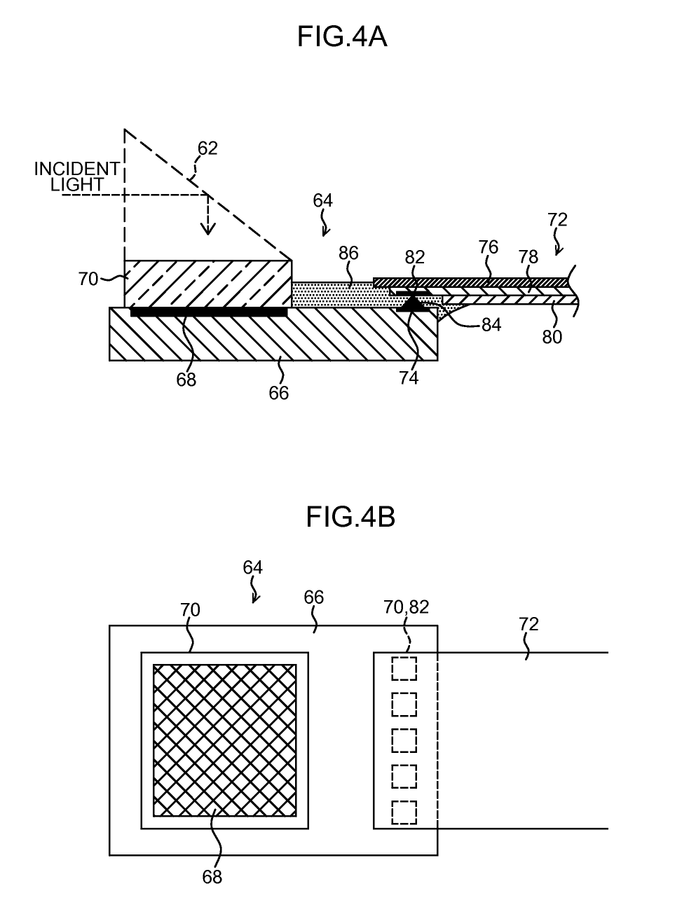

Subsequently, the internal structure of the distal end portion 44 will be described. FIG. 3 is an outline diagram illustrating an essential part of the internal structure of the distal end portion 44. As shown in FIG. 3, inside the distal end portion 44, an objective lens group 60 including a plurality of lenses 60a to 60c that collects subject light (incident light) taken in through the observation window 52 is disposed, and behind them, a prism 62 that converts an optical path of the subject light by 90 degrees is disposed. At a lower end of the prism 62, an image pickup module 64 is arranged, and the subject light whose optical path is converted by the prism 62 by 90 degrees is formed on the image pickup surface (not shown in FIG. 3 and described as reference numeral 68 in FIG. 4) of the image pickup module 64.

Here, a configuration of the image pickup module 64 used in this embodiment will be described in detail. FIG. 4 is a configuration diagram illustrating a detail of the image pickup module 64 incorporated in the distal end portion 44, in which FIG. 4A is a side sectional view and FIG. 4B is a plan view.

As shown in FIGS. 4A and 4B, the image pickup module 64 of this embodiment mainly includes a solid-state image pickup element chip 66 in which a solid-state image pickup element (such as a CCD (Charge Coupled Device) image pickup element, CMOS (Complementary Metal-Oxide Semiconductor) image pickup element and the like) is disposed on a semiconductor board made of silicone, for example, a cover glass 70 that is arranged between the solid-state image pickup element chip 66 and the prism 62 and covers the image pickup surface (light receiving portion) 68 of the solid-state image pickup element chip 66, and a flexible board (FPC (Flexible Printed Circuit) board) 72 whose one end is connected to the solid-state image pickup element chip 66.

On a principal surface of the solid-state image pickup element chip 66, the image pickup surface 68 is arranged substantially at the center part, and a plurality of electrode pads 74 for input/output of signals to/from the image pickup surface 68 are disposed on the peripheral part thereof. On each electrode pad 74, a bump 84 is fastened, respectively.

The flexible board 72 mainly includes a base layer (base material) 76 having insulation and flexibility, a wiring pattern 78 formed on the surface of the base layer 76, and a cover layer (protective layer) 80 that covers the surface of the base layer 76 on which the wiring pattern 78 is formed. The flexible board 72 is arranged so that the cover layer 80 is directed to the solid-state image pickup element chip 66 side (lower side in FIG. 4).

As an example of a constituting material of the flexible board 72, a polyimide film is preferably used for the base layer 76, a copper foil pattern for the wiring pattern 78, and a polyimide coverlay for the cover layer 80, respectively. The cover layer 80 is formed by a material having insulation and flexibility similarly to the base layer 76. The base layer 76 and the cover layer 80 may be formed by the same material or by different materials.

On one end of the flexible board 72 (end portion on the solid-state image pickup element chip 66 side), a plurality of connection terminal portions 82 that electrically connect to the electrode pads 74 of solid-state image pickup element chip 66 are mounted. The connection terminal portion 82 is a portion formed on the end portion of the wiring pattern 78 drawn on the base layer 76 and exposed to the surface without being covered by the cover layer 80.

In this embodiment, in order to improve workability when the solid-state image pickup element chip 66 is mounted on the flexible board 72 and to prevent bending of the connection terminal portion (lead portion) 82, it is configured such that the base layer 76 is present at least at a position where the connection terminal portion 82 is formed. That is, it is configured such that a mounting portion of the solid-state image pickup element chip 66 in the flexible board 72 does not become a flying lead structure.

The end portions of the solid-state image pickup element chip 66 and the flexible board 72 have an overlap structure in which they are overlapped with each other. In other words, the image pickup module 64 includes a overlap portion in which the solid-state image pickup element chip 66 and the flexible board 72 overlap each other. The electrode pad 74 of the solid-state image pickup element chip 66 and the connection terminal portion 82 of the flexible board 72 are electrically connected to each other through the bump 84.

Also, in order to ensure the connection strength between the end portions of the solid-state image pickup element chip 66 and the flexible board 72, the peripheral portions of the electrode pad 74 and the connection terminal portion 82 are sealed and fixed by a sealing resin (thermosetting resin) 86. As the sealing resin 86, ACP/NCP resin (anisotropically conductive paste/nonconductive paste resin) is used, and epoxy resin or silicone resin, for example, are suitable. Also, instead of the ACP/NCP resin, an ACF/NCF film (anisotropically conductive film/nonconductive film) may be used.

Though not shown, a similar connection terminal portion is disposed also on the other end of the flexible board 72. To this connection terminal portion, a signal transmission cable for transmission/reception of a signal with the processor 26 is electrically connected. The signal transmission cable is inserted through the insertion portion 12, the hand operation portion 14, the universal cable 16 and the like of FIG. 1 and extended to the electric connector 24 and connected to the processor 26. The signal transmission cable supplies power to the solid-state image pickup element chip 66 and electronic components and the like (not shown) mounted on the flexible board 72 and transmits an electric signal photoelectrically converted by the solid-state image pickup element chip 66 to the processor 26.

By means of the above configuration, the subject light taken in through the observation window 52 in the distal end portion 44 is collected by the objective lens group 60 and has the direction of the optical path thereof converted by the prism 62 by 90 degrees and then, formed on the image pickup surface 68 of the image pickup module 64. Then, the electric signal (image pickup signal) of the subject light photoelectrically converted by the image pickup module 64 is outputted to the processor 26 through the flexible board 72 and the signal transmission cable and converted to a video signal in the processor 26. As a result, an observation image (endoscopic image) is displayed on the monitor device 50 connected to the processor 26.

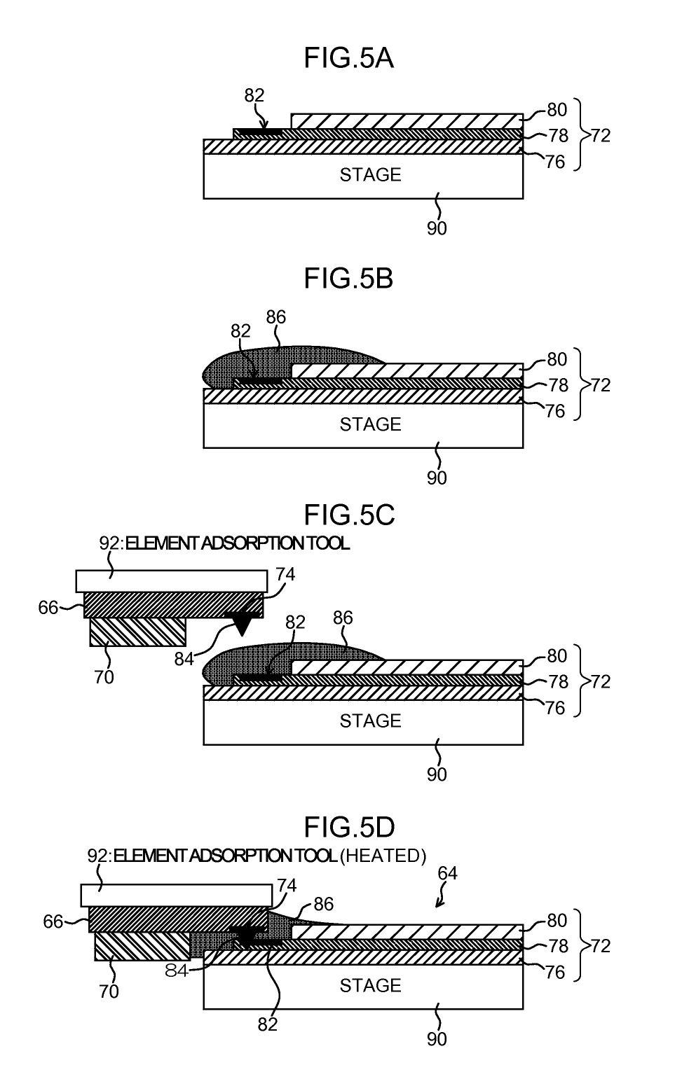

Subsequently, a manufacturing method of the image pickup module 64 of this embodiment will be described. FIGS. 5A to 5D are explanatory diagrams illustrating an example of the manufacturing method of the image pickup module 64.

First, as shown in FIG. 5A, the flexible board 72 composed of the base layer 76, the wiring pattern 78, and the cover layer 80 is set on a stage 90. At this time, the board is set so that the base layer 76 of the flexible board 72 is directed to the stage 90 side. Also, the connection terminal portion 82 formed on one end of the wiring pattern 78 is exposed to the surface.

Subsequently, as shown in FIG. 5B, the sealing resin 86 is applied to the peripheral portion of the connection terminal portion 82 formed on one end of the flexible board 72 set on the stage 90. As described above, as the sealing resin 86, the ACP/NCP resin (anisotropically conductive paste/nonconductive paste resin) is used. Instead of the ACP/NCP resin, an ACF/NCF film (anisotropically conductive film/nonconductive paste film) may be bonded.

Then, as shown in FIG. 5C, the solid-state image pickup element chip 66 to which the cover glass 70 is joined is adsorbed and fixed by a predetermined tool (element adsorption tool) 92, and the electrode pad 74 of the solid-state image pickup element chip 66 and the connection terminal portion 82 of the flexible board 72 are aligned so that the end portions of the solid-state image pickup element chip 66 and the flexible board 72 are overlapped with each other.

After the above mentioned alignment is completed, as shown in FIG. 5D, the bump 84 arranged on the electrode pad 74 of the solid-state image pickup element chip 66 and the connection terminal portion 82 of the flexible board 72 are bonded, and the element adsorption tool 92 is heated. As a result, heat energy is applied from the element adsorption tool 92 to the sealing resin 86 through the solid-state image pickup element chip 66, and the sealing resin 86 is cured.

Since the solid-state image pickup element chip 66 contains a resin material such as a color filter, micro lens and the like, if a heating temperature applied when the solid-state image pickup element chip 66 and the flexible board 72 are connected is too high, the resin material is deteriorated, and the solid-state image pickup element might be broken.

Thus, in this embodiment, as the sealing resin 86, a thermosetting resin that can be cured at a temperature at which the solid-state image pickup element is thermally deteriorated or below is used. Specifically, a bonding temperature condition is 180° C./10 sec, and a low-temperature thermosetting resin (for example, produced by Henkel AG & Co. KGaA, product no. FP5110) is preferably used. As a result, thermal deterioration of the solid-state image pickup element is prevented.

After the sealing resin 86 is cured as above, the adsorption and fixation of the solid-state image pickup element chip 66 by the element adsorption tool 92 is released, and the flexible board 72 is removed from the stage 90 so that the image pickup module 64 of this embodiment is completed.

According to the image pickup module 64 of this embodiment, the end portions of the solid-state image pickup element chip 66 and the flexible board 72 have an overlap structure (overlap portion) in which they are overlapped with each other, and the electrode pad 74 formed on the solid-state image pickup element chip 66 and the connection terminal portion 82 formed on the flexible board 72 are electrically connected to each other through the bump 84. That is, the solid-state image pickup element chip 66 has an extended arrangement structure in which the chip 66 is protruded and extended outward from the end portion of the flexible board 72, and the solid-state image pickup element chip 66 and the flexible board 72 are electrically connected to each other directly without interposing an intermediate connection member between them (such as a wire by wire bonding or a board for connection). As a result, the size of the image pickup module 64 can be reduced, and reliability of the electric connection and the electric noise resistance can be improved by reduction of the numbers of components and the connection spots.

Also, in this embodiment, the mounting portion of the solid-state image pickup element chip 66 in the flexible board 72 (that is, the connection terminal portion 82) does not have a flying lead structure, and rigidity can be ensured by the base layer 76 located on the board back-face portion, whereby the workability at connection or resin sealing is improved and bending of the mounting portion can be prevented.

The image pickup module, the manufacturing method thereof, and the endoscopic device of the presently disclosed subject matter have been described in detail, but the presently disclosed subject matter is not limited to the above embodiment, but it is needless to say that various improvements and variations can be made within a range not departing from the gist of the presently disclosed subject matter.

What is claimed is:

1. An image pickup module, comprising:
a solid-state image pickup element chip having an image pickup surface;
a cover glass that covers the image pickup surface;
a wiring board on which the solid-state image pickup element chip is mounted;
a overlap portion in which an end portion of the solid-state image pickup element chip and an end portion of the wiring board overlap each other; and
a connection terminal portion configured to electrically connect a first electrode portion formed on the end portion of the solid-state image pickup element chip and a second electrode portion formed on the end portion of the wiring board through a bump.

2. The image pickup module according to claim 1, wherein
the end portions of the solid-state image pickup element chip and the wiring board are sealed and fixed to each other by a resin.

3. The image pickup module according to claim 1, wherein
the first electrode is formed on a same plane as the image pickup surface.

4. The image pickup module according to claim 1, wherein
the wiring board is a flexible board having flexibility.

5. The image pickup module according to claim 1, wherein
the flexible board includes at least a base layer, a wiring pattern formed on the base layer, and a cover layer that covers a surface of the base layer on which the wiring pattern is formed.

6. A manufacturing method of an image pickup module including a solid-state image pickup element chip having an image pickup surface, a cover glass that covers the image pickup surface, and a wiring board on which the solid-state image pickup element chip is mounted, comprising:
aligning an end portion of the solid-state image pickup element chip and an end portion of the wiring board to overlap each other; and
electrically connecting, after the alignment, a first electrode portion formed on the end potion of the solid-state image pickup element chip and a second electrode portion formed on the end portion of the wiring board through a bump.

7. The manufacturing method of an image pickup module, according to claim 6, comprising:
applying a thermosetting resin on at least one of the end portions of the solid-state image pickup element chip and the wiring board; and
heating and curing the thermosetting resin in a state in which the first electrode portion and the second electrode portion are electrically connected to each other through the bump.

8. The manufacturing method of an image pickup module, according to claim 7, wherein
the heating of the thermosetting resin is performed indirectly through a tool that adsorbs the solid-state image pickup element chip.

9. The manufacturing method of an image pickup module, according to claim 7, wherein
the thermosetting resin is a low-temperature thermosetting resin that is cured at a temperature at which a solid-state image pickup element of the solid-state image pickup element chip is thermally deteriorated or below.

10. An endoscopic device including the image pickup module according to claim 1.
